(19) United States
(12) Patent Application Publication
(10) Pub. No.: US 2025/0331785 A1
(43) Pub. Date: Oct. 30, 2025

(54) PEDAL ASSEMBLY, CONTROL METHOD THEREFOR, AND MEDICAL IMAGING SCANNING BED ASSEMBLY

(71) Applicant: GE Precision Healthcare LLC, Waukesha, WI (US)

(72) Inventors: Yifei Dong, Beijing (CN); Yigang Xia, Beijing (CN); Qian Xu, Beijing (CN)

(21) Appl. No.: 19/192,022

(22) Filed: Apr. 28, 2025

(30) Foreign Application Priority Data
Apr. 28, 2024 (CN) 202410520570.7

(51) Int. Cl. A61B 6/04; A61B 6/00; A61B 6/03

(52) U.S. Cl. CPC A61B 6/0407; A61B 6/035; A61B 6/54

(57) ABSTRACT

A pedal assembly, a control method therefor, and a medical imaging scanning bed assembly are described herein. The pedal assembly includes a pedal, having a first end portion close to an inner side of the pedal assembly in a first direction and a second end portion away from the inner side of the pedal assembly; a switch, connected to the pedal, and being in a triggered state when the pedal leaves a predetermined position in a second direction; a first sensor, detecting, via a through hole on a surface of the pedal, whether a first region of the surface of the pedal is obstructed; and a controller, outputting a control signal based on a detection result of the first sensor and a state of the switch. The present application can prevent unexpected movement of a mechanism, such as a scanning bed, caused by an operator mistakenly pressing the pedal.

### CROSS-REFERENCE TO RELATED APPLICATIONS

This application claims priority to Chinese Application No. 202410520570.7, filed on Apr. 28, 2024, the disclosure of which is incorporated herein by reference in its entirety.

### TECHNICAL FIELD

Embodiments of the present application relate to the technical field of medical devices, and in particular, to a pedal assembly, a control method therefor, and a medical imaging scanning bed assembly.

### BACKGROUND

A medical imaging device is configured to scan a patient in a non-invasive manner to acquire a medical image of an anatomical tissue of interest of the patient, thereby assisting doctors in making a diagnosis. For example, computed tomography (CT) utilizes accurately collimated X-ray beams or gamma rays, together with a highly sensitive detector, to perform cross-sectional scans one by one around a certain site of a human body, is characterized by a short scanning time, image clarity, and the like, and can be used to detect a wide range of diseases.

CT tomography has many advantages. First, CT tomography can obtain images having high density and resolution, so that the doctors can clearly observe information such as the size, shape, and position of a site of a lesion. Second, CT scanning is non-invasive to the human body and has a relatively low radiation dose, thereby offering a high level of safety for patients. In addition, CT scanning has a high speed and is suitable for scenarios requiring rapid diagnosis, such as an emergency treatment.

CT tomography has a wide range of clinical applications. In the diagnosis of head and neck diseases, CT scanning can help doctors observe the skull and soft tissue structures to locate diseased tissues. In the diagnosis of thoracic diseases, CT scanning can be used to observe lung and heart structures, to detect diseases such as lung cancer, pneumonia, and pneumothorax. In the diagnosis of abdominal diseases, CT scanning can be used to observe the structures of organs such as the liver, spleen, and kidney, so as to detect diseases such as tumors and calculi. In addition, CT tomography may also play a role in the diagnosis of bone and spinal diseases.

It should be noted that the above introduction of the background is only for the convenience of clearly and completely describing the technical solutions of the present application, and for the convenience of understanding for those skilled in the art.

### SUMMARY

A medical imaging device such as a CT device has a scanning bed (which may also be referred to as a patient table) and a pedal assembly. A subject under examination is placed on the scanning bed, and an operator of the medical imaging device can control the scanning bed to perform lifting or lowering movement by operating the pedal assembly (for example, stepping down on a pedal in the pedal assembly).

The inventors have found that the operator sometimes may unintentionally step on the pedal during the operation of the medical imaging device, causing the scanning bed to lift or lower unexpectedly, which not only affects a detection effect for the subject under examination, but also sometimes causes a risk of personal injury or device damage. Therefore, how to prevent unexpected movement of the scanning bed occurring when the operator mistakenly steps on the pedal is a problem to be resolved.

In view of at least one of the technical problems described above or other similar problems, embodiments of the present application provide a pedal assembly, a control method therefor, and a medical imaging scanning bed assembly. The pedal assembly uses a first sensor to detect whether a predetermined position of a pedaling surface of a pedal is obstructed, and outputs a control signal based on a detection result of the first sensor and a state of a switch, wherein the control signal may be used to control the operation of a mechanism such as a scanning bed. In this way, unexpected movement of a mechanism such as a scanning bed occurring when the operator mistakenly steps on the pedal is avoided.

According to one aspect of the embodiments of the present application, provided is a pedal assembly. The pedal assembly includes a pedal, having a first end portion close to an inner side of the pedal assembly in a first direction and a second end portion away from the inner side of the pedal assembly, a switch, connected to the pedal, the switch being in a triggered state when the pedal leaves a predetermined position in a second direction, a first sensor, detecting, via a through hole on a pedaling surface of the pedal, whether a first region of the pedaling surface of the pedal is obstructed, and a controller, outputting a control signal based on a detection result of the first sensor and a state of the switch.

According to another aspect of the embodiments of the present application, provided is a medical imaging scanning bed assembly. The medical imaging scanning bed assembly comprises a medical imaging scanning bed and the pedal assembly according to any one of the embodiments described above, a controller of the pedal assembly outputting a control signal to control movement of the medical imaging scanning bed.

According to another aspect of the embodiments of the present application, provided is a control method based on a pedal assembly. The control method includes outputting, by a controller, a control signal based on a detection result of a first sensor and a state of a switch.

One of the beneficial effects of the embodiments of the present application is that: whether a predetermined position of the pedaling surface of the pedal is obstructed is detected by means of the first sensor, and the control signal is outputted based on the detection result of the first sensor and the state of the switch, wherein the control signal may be used to control the operation of a mechanism such as a scanning bed. For example, when an operator unintentionally steps on the pedal and causes the switch to be in a triggered state, the predetermined position of the pedaling surface of the pedal is not obstructed by a foot or shoe of the operator, and the controller does not output a control signal that causes the mechanism such as the scanning bed to operate. In this way, unexpected movement of the mechanism such as the scanning bed is avoided.

With reference to the following description and drawings, specific implementations of the embodiments of the present application are disclosed in detail, and the way in which the principles of the embodiments of the present application can be employed is illustrated. It should be understood that the embodiments of the present application are not limited in scope thereby. Within the scope of the spirit and clauses of the appended claims, the embodiments of the present application comprise many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and those of ordinary skill in the art may obtain other implementations according to the drawings without involving inventive effort. In the drawings:

FIG. 1 is a schematic diagram of a CT device according to an embodiment of the present application;

FIG. 2 is a schematic diagram of a CT imaging system according to an embodiment of the present application;

FIG. 3 is a schematic diagram of a pedal assembly according to an embodiment of the present application;

FIG. 4 is a schematic diagram of a connecting circuit of a first sensor 33, a switch 32, and a controller 34 according to the present application;

FIG. 5 is a schematic diagram of a pedal in FIG. 3;

FIG. 6 is another schematic diagram of a pedal assembly according to an embodiment of the present application;

FIG. 7 is a schematic diagram of a pedal in FIG. 6;

FIG. 8 is a schematic diagram of a connecting circuit of a first sensor 33, a second sensor 37, a switch 32, and a controller 34a according to the present application; and

FIG. 9 is a schematic diagram of a control method based on a pedal assembly according to an embodiment of the present application.

### DETAILED DESCRIPTION

The aforementioned and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first”, “second”, etc., are used to distinguish different elements with respect to naming, but do not represent a spatial arrangement or temporal order, etc., of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a”, “the”, etc., include plural forms, and should be broadly construed as “a type of” or “a class of” rather than being limited to the meaning of “one”. In addition, the term “the” should be construed as including both the singular and plural forms, unless otherwise specified in the context. In addition, the term “according to” should be construed as “at least in part according to . . . ” and the term “based on” should be construed as “based at least in part on . . . ”, unless otherwise specified in the context.

The features described and/or illustrated for one implementation may be used in one or more other implementations in the same or similar way, be combined with features in other embodiments, or replace features in other implementations. The terms “include/comprise” when used herein refer to the presence of features, integrated components, steps, or assemblies, but do not preclude the presence or addition of one or more other features, integrated components, steps, or assemblies.

The medical imaging device described in the present application is applicable to various medical imaging modalities, and includes, but is not limited to, computed tomography (CT) devices, or positron emission tomography (PET)-CT, magnetic resonance imaging (MRI), or any other suitable medical imaging devices.

The system obtaining the medical imaging data may include the aforementioned medical imaging device, may include a separate computer device connected to the medical imaging device, and may further include a computer device connected to an Internet cloud, the computer device being connected by means of the Internet to the medical imaging device or a memory for storing medical images. The imaging method may be independently or jointly implemented by the aforementioned medical imaging device, the computer device connected to the medical imaging device, and the computer device connected to the Internet cloud. For example, the system obtaining the medical image data may be a CT imaging system, etc.

As an example, the embodiments of the present application are described below in conjunction with an X-ray computed tomography (CT) imaging device. Those skilled in the art would appreciate that the embodiments of the present application can also be applied to other medical imaging devices.

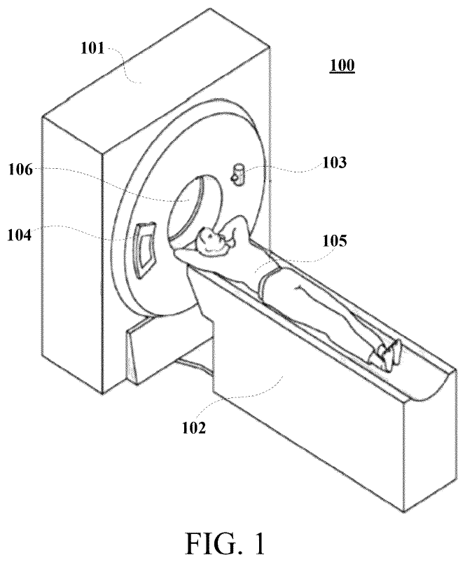

FIG. 1 is a schematic diagram of a CT device according to an embodiment of the present application, and schematically shows a CT device 100. As shown in FIG. 1, the CT device 100 includes a scanning gantry 101 and a patient table 102. The scanning gantry 101 has an X-ray source 103, and the X-ray source 103 projects an X-ray beam toward a detector assembly or collimator 104 on an opposite side of the scanning gantry 101. A subject under examination 105 can lie flat on the patient table 102 and be moved into a scanning gantry opening 106 along with the patient table 102. Medical image data of the subject under examination 105 can be obtained by scanning performed by the X-ray source 103.

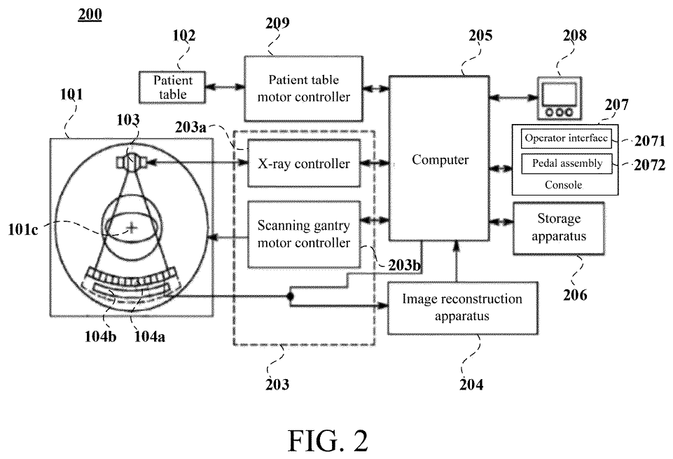

FIG. 2 is a schematic diagram of a CT imaging system according to an embodiment of the present application, and schematically shows a block diagram of a CT imaging system 200. As shown in FIG. 2, the detector assembly 104 includes a plurality of detector units 104a and a data acquisition system (DAS) 104b. The plurality of detector units 104a sense a projected X-ray passing through the subject under examination 105.

The DAS 104b, according to the sensing of the detector units 104a, converts collected information into projection data for subsequent processing. During the scanning for acquiring the X-ray projection data, the scanning gantry 101 and components mounted thereon rotate around a center of rotation 101c.

The rotation of the scanning gantry 101 and the operation of the X-ray source 103 are controlled by a control mechanism 203 of the CT imaging system 200. The control mechanism 203 includes an X-ray controller 203a that provides power and a timing signal to the X-ray source 103 and a scanning gantry motor controller 203b that controls the rotational speed and position of the scanning gantry 101. An image reconstruction apparatus 204 receives the projection data from the DAS 104b and performs image reconstruction. A reconstructed image is transmitted as an input to a computer 205, and the computer 205 stores the image in a mass storage apparatus 206.

The computer 205 also receives commands and scanning parameters from an operator by means of a console 207. The console 207 has an operator interface 2071 in a certain form, for example, a keyboard, a mouse, or a voice activated controller. The console 207 may also have an input apparatus such as a pedal assembly 2072, wherein an operator may step on or press a pedal in the pedal assembly with a part of the body (e.g., a foot) to input instructions for moving (e.g., lifting or lowering) the patient table 102. In addition, the console 207 may have another suitable input apparatus. An associated display 208 allows the operator to observe a reconstructed image and other data from the computer 205. The commands and parameters provided by the operator are used by the computer 205 to provide control signals and information to the DAS 104b, the X-ray controller 203a, and the scanning gantry motor controller 203b. Additionally, the computer 205 operates a patient table motor controller 209 which controls the patient table 102 so as to position the subject under examination 105 and the scanning gantry 101. In particular, the patient table 102 moves the subject under examination 105 to, fully or in part, pass through the scanning gantry opening 106 in FIG. 1.

The device and system for acquiring medical image data (which may also be referred to as medical images or medical image data) according to the embodiments of the present application are schematically described above, but the present application is not limited thereto. The medical imaging device may be a CT device, or a PET-CT or any other suitable imaging device. A storage device may be located within the medical imaging device, in a server outside the medical imaging device, in an independent medical imaging storage system (such as a Picture Archiving and Communication System (PACS)), and/or in a remote cloud storage system.

In addition, a medical imaging workstation may be provided locally to the medical imaging device, that is, the medical imaging workstation is provided close to the medical imaging device, and the two may both be located in a scanning room, an imaging department, or the same hospital. In contrast, a medical image cloud platform analysis system may be positioned distant from the medical imaging device, e.g., arranged at a cloud end that is in communication with the medical imaging device.

As an example, after a medical institution completes an imaging scan using the medical imaging device, data obtained by scanning is stored in a storage device. A medical imaging workstation may directly read the data obtained by scanning and perform image processing by means of a processor thereof. As another example, the medical image cloud platform analysis system may read a medical image in the storage device by means of remote communication to provide “software as a service (SaaS)”. SaaS can exist between hospitals, between a hospital and an imaging center, or between a hospital and a third-party online diagnosis and treatment service provider.

Medical image scanning is schematically illustrated above, and the embodiments of the present application are described in detail below in view of the accompanying drawings. In the embodiments described below, the medical imaging device being a CT device is used as an example for description, and the content of the description is also applicable to other medical imaging devices.

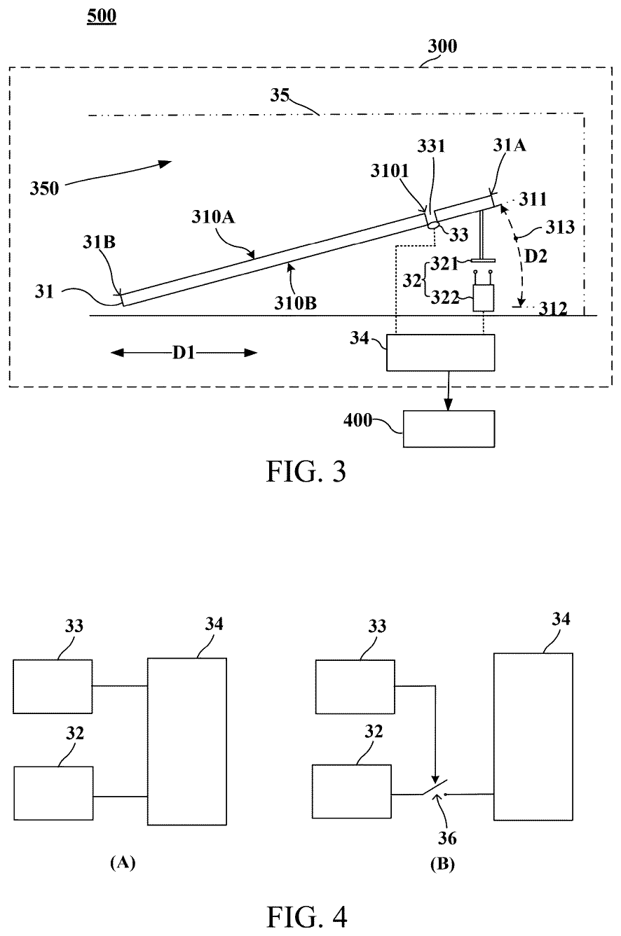

Provided in the embodiments of the present application is a pedal assembly. FIG. 3 is a schematic diagram of a pedal assembly according to an embodiment of the present application. As shown in FIG. 3, the pedal assembly 300 includes: a pedal 31, a switch 32, a first sensor 33, and a controller 34. As shown in FIG. 3, the pedal 31 has a first end portion 31A close to an inner side of the pedal assembly 300 in a first direction D1 and a second end portion 31B away from the inner side of the pedal assembly 300.

The pedal 31 leaves a predetermined position in a second direction D2 when stepped on or pressed by an operator, and the pedal 31 returns in the second direction D2 to the predetermined position when the stepping or pressing by the operator disappears. For example, the pedal 31 has a restoring force providing mechanism (not shown) capable of providing a restoring force for the pedal 31, so that the pedal 31 returns in the second direction D2 to the predetermined position when the stepping or pressing by the operator disappears. The restoring force providing mechanism may, for example, have an element such as a spring.

As shown in FIG. 3, the predetermined position of the pedal 31 may correspond to a predetermined range in a stroke of the pedal 31 in the second direction D2. For example, the stroke of the pedal 31 in the second direction D2 has a start position 311 and an end position 312, wherein the pedal 31 is located at the start position 311 when not stepped on or pressed by the operator, and the pedal 31 is moved in the second direction D2 to the end position 312 at most when stepped on or pressed by the operator. In the stroke of the pedal 31 in the second direction D2, there is a switch triggering position 313.

A range from the start position 311 to the switch triggering position 313 may be the predetermined range described above, that is: when the pedal 31 is in the range from the start position 311 to the switch triggering position 313, the pedal 31 is located at the predetermined position, and when the pedal 31 is moved to a range from the switch triggering position 313 to the end position 312, the pedal 31 leaves the predetermined position. In the second direction D2, the distance from the start position 311 to the switch triggering position 313 may be 15% to 20%, or another value range, of the distance from the start position 311 to the end position 312.

In the present application, the switch 32 is connected to the pedal 31, and the switch 32 is in a triggered state when the pedal 31 leaves the predetermined position in the second direction D2. In some examples, the switch 32 may have a movable portion 321 and a fixed portion 322, the movable portion 321 moves together with the pedal 31, and when the pedal 31 leaves the predetermined position, the movable portion 321 and the fixed portion 322 come into contact with each other, and the switch 32 is in the triggered state. In addition, the structure of the switch 32 may not be limited thereto. For example, in some other examples, the switch 32 may have a displacement detecting element and a signal generating element, wherein the displacement detecting element may detect movement displacement of the pedal 31 to determine whether the pedal 31 has left the predetermined position, and when it is determined that the pedal 31 has left the predetermined position, the displacement detecting element causes the signal generating element to generate a corresponding state signal, to indicate that the switch 32 is in the triggered state. A mounting position of the switch 32 is not limited to being close to one side of the first end portion 31A of the pedal 31, as shown in FIG. 3. In another embodiment, the switch 32 may alternatively be disposed at a position close to the second end portion 31B of the pedal 31.

As shown in FIG. 3, the first sensor 33 may detect, via a through hole 331 on a pedaling surface 310A of the pedal 31, whether a first region 3101 of the pedaling surface 310A of the pedal 31 is obstructed. For example, when a part of the body of the operator (for example, a foot) steps on the pedaling surface 310A of the pedal 31, and a stepped-on region includes the first region 3101, the first sensor 33 may detect that the first region 3101 is obstructed. For another example, when a part of the body of the operator (for example, a foot) steps on the pedaling surface 310A of the pedal 31 but the stepped-on region does not include the first region 3101, the first sensor 33 does not detect that the first region 3101 is obstructed or the first sensor 33 detects that the first region 3101 is not obstructed. The pedaling surface 310A is an upper surface or an outer surface of the pedal 31, to be stepped on or pressed by a part of the body of the operator (for example, a foot).

In the present application, the first sensor 33 may be mounted in a space on a side of a back surface 310B of the pedal 31. The back surface 310B is a surface opposite to the pedaling surface 310A of the pedal 31, and the back surface 310B faces the switch 32, and may serve as a non-pedaling surface or an inner surface of the pedal 31.

For example, the first sensor 33 may be directly mounted on the back surface 31B of the pedal 31, as shown by a solid line in FIG. 3. For another example, as shown by a dashed line in FIG. 3, the first sensor 33 is mounted outside the pedal 31 (i.e., the first sensor 33 is not directly connected to the pedal 31) at a distance from the back surface 31B, wherein the distance between the first sensor 33 and the back surface 310B of the pedal 31 is smaller than the distance between the first sensor 33 and the pedaling surface 310A of the pedal 31.

Therefore, the space on the side of the back surface 310B of the pedal 31 can be fully utilized, thereby avoiding enlargement and complexity of the pedal assembly 300. In addition, the introduction of the first sensor 33 has little impact on the use of the pedal 31. For example, if the first sensor 33 was disposed directly on the pedaling surface 310A of the pedal 31, the operator would be easily hindered by the first sensor 33 when stepping on the pedal, which would affect the use experience and convenience of use. In addition, disposing the first sensor 33 based on the position provided in the present application can expand the scope of application of the pedal assembly 300. For example, in the present application, the first sensor 33 is mounted in the space on the side of the back surface 310B of the pedal 31. In this way, the first sensor 33 can be mounted without depending on a protective cover 35 of the pedal 31, that is, the first sensor 35 can be mounted in the space on the side of the back surface 310B of the pedal 31 regardless of whether the protective cover 35 is present above the pedal 31, whereas if the first sensor 33 is mounted on the protective cover 35 (for example, the first sensor 33 is mounted on the top or a side wall of the protective cover), the pedal assembly can be applied only to a scenario with the protective cover 35, but cannot be applied to a scenario without the protective cover 35.

In the present application, the first sensor 33 may be a photoelectric sensor, and in some examples, the first sensor 33 is a reflective photoelectric sensor, wherein light emitted from the first sensor 33 passes through the through hole 331 and light reflected by an object is received via the through hole 331, thereby determining whether the first region 3101 is obstructed. For example, when a region of the pedaling surface 310A of the pedal 31, stepped on by a foot of the operator, includes the first region 3101, light reflected by the foot of the operator is received by the first sensor 33, and therefore it is determined that the first region 3101 is obstructed.

In addition, the photoelectric sensor is merely an example, and the present application is not limited thereto. The first sensor 33 may alternatively be a capacitive sensor, an image recognition sensor, a pressure sensor, a magnetic sensor, or the like.

In the present application, when the first sensor 33 detects that the first region 3101 of the pedaling surface 310A of the pedal 31 is obstructed, it may correspond to a case in which a part of the body of the operator (for example, a foot) intentionally steps on or presses the pedal 31, and when the first sensor 33 does not detect that the first region 3101 of the pedaling surface 310A of the pedal 31 is obstructed, it may correspond to a case in which a part of the body of the operator (for example, a foot) does not intentionally step on or press the pedal 31.

In the present application, as shown in FIG. 3, the controller 34 outputs a control signal based on a detection result of the first sensor 33 and a state of the switch 32, wherein the control signal may be used to control an external mechanism 400 to move or not to move. Therefore, the controller 34 outputs the control signal based on not only the state of the switch 32 but also the detection result of the first sensor 33 (i.e., whether the pedaling surface 310A of the pedal 31 is obstructed), that is, the controller 34 outputs the control signal based on more factors including the state of the switch 32, so that it is possible to avoid unexpected movement of the external mechanism 400 occurring when a part of the body of the operator (for example, a foot) unintentionally touches the pedal 31, thereby improving the stability and safety of the movement of the external mechanism 400.

For example, in some scenarios, when the switch 32 is in the triggered state and the first sensor 33 detects that the first region 3101 is obstructed, the controller 34 outputs a control signal for allowing movement, wherein the control signal for allowing movement may control the external mechanism 400 to move. These scenarios correspond to a case in which a part of the body of the operator (for example, a foot) intentionally steps on or presses the pedal 31 and the pedal 31 leaves the predetermined position.

For another example, in some other scenarios, when the switch 32 is in the triggered state and the first sensor 33 detects that the first region 3101 is not obstructed, the controller 34 does not output a control signal for allowing movement, wherein not outputting a control signal for allowing movement may be not outputting any signal, thereby causing the external mechanism 400 to remain in a stationary state, or outputting a control signal to cause the external mechanism to remain in the stationary state. These scenarios correspond to a case in which a part of the body of the operator (for example, a foot) unintentionally steps on or presses the pedal 31 and the pedal 31 leaves the predetermined position.

For still another example, in some scenarios, when the switch 32 is in an untriggered state, the controller 34 does not output a control signal for allowing movement. These scenarios correspond to a case in which a part of the body of the operator (for example, a foot) does not step on or press the pedal 31 or steps on or presses the pedal 31 but the pedal 31 does not leave the predetermined position.

In the present application, the external mechanism 400 controlled by the controller 34 may include a scanning bed that, for example, is applied to a medical imaging system, wherein the medical imaging system has, for example, a CT (computed tomography) imaging device, or a PET (positron emission tomography)-CT, a magnetic resonance imaging (MRI), or any other suitable medical imaging device. Therefore, the scanning bed may also be referred to as a medical imaging scanning bed. Therefore, the present application may provide a medical imaging scanning bed assembly 500 including the external mechanism 400 and the pedal assembly 300.

For example, in the medical imaging scanning bed assembly 500, portions other than the controller 34 in the pedal assembly 300 may correspond to the pedal assembly 2072 of FIG. 2. The controller 34 of the pedal assembly 300 corresponds to the computer 205 of FIG. 2, and the external mechanism 400 may correspond to the patient table 102 of FIG. 2, i.e., a medical imaging scanning bed. For example, the controller 34 (corresponding to the computer 205 of FIG. 2) of the pedal assembly 300 outputs the control signal based on the detection result of the first sensor and the state of the switch, the control signal is inputted to the patient table motor controller 209 of FIG. 2, and the patient table motor controller 209 outputs a signal to the patient table 102 (corresponding to the external mechanism 400 of FIG. 3), so that the patient table 102 moves (e.g., lifts or lowers) or does not move.

In addition, in another example, the controller 34 of the pedal assembly 300 may alternatively correspond to both the computer 205 and the patient table motor controller 209 of FIG. 2, that is, the controller 34 (corresponding to both the computer 205 and the patient table motor controller 209 of FIG. 2) of the pedal assembly 300 outputs the control signal based on the detection result of the first sensor and the state of the switch, and the control signal is inputted to the patient table 102 (corresponding to the external mechanism 400 of FIG. 3) of FIG. 2, so that the patient table 102 moves (e.g., lifts or lowers) or does not move.

In the medical imaging scanning bed assembly 500 of the present application when a part of the body of the operator (for example, a foot) intentionally steps on or presses the pedal 31 and the pedal 31 leaves the predetermined position, the switch 32 is in the triggered state and the first sensor 33 detects that the first region 3101 is obstructed, so that the controller 34 of the pedal assembly 300 outputs the control signal for allowing movement of the patient table 102 to cause the patient table 102 performs lifting or lowering movement. When a part of the body of the operator (for example, a foot) unintentionally steps on or presses the pedal 31 and the pedal 31 leaves the predetermined position, the switch 32 is in the triggered state and the first sensor 33 detects that the first region 3101 is not obstructed, so that the controller 34 of the pedal assembly 300 does not output the control signal for allowing movement of the patient table 102, and therefore, the patient table 102 remains stationary or stops lifting or lowering movement, thereby preventing unexpected movement of the patient table 102 due to an erroneous operation by the operator.

When a part of the body of the operator (for example, a foot) does not step on or press the pedal 31 or steps on or presses the pedal 31 but the pedal 31 does not leave the predetermined position, the switch 32 is in the untriggered state, and therefore, the patient table 102 remains stationary or stops lifting or lowering movement.

In the present application, the pedal assembly 300 being applied to the medical imaging scanning bed assembly 500 is used as an example for description. However, the description is also applicable to a case in which the pedal assembly 300 is applied to another system other than the medical imaging scanning bed assembly 500.

FIG. 4 is a schematic diagram of a connecting circuit of the first sensor 33, the switch 32, and the controller 34 according to the present application. (A) and (B) of FIG. 4 may be different embodiments.

In the embodiment shown by (A) of FIG. 4: the first sensor 33 is connected to the controller 34, and the first sensor 33 outputs a signal indicating a detection result to the controller 34. The switch 32 is connected to the controller 34, and the switch 32 outputs a signal indicating a state of the switch to the controller 34. The controller 34 outputs a control signal based on the signal outputted by the first sensor 33 and indicating the detection result and the signal outputted by the switch 32 and indicating the state of the switch. For logic for the controller 34 to output the control signal, reference may be made to the description described above with respect to FIG. 3. The connecting circuit in the embodiment shown by (A) of FIG. 4 is simple.

In the embodiment shown by (B) of FIG. 4: the first sensor 33 is connected to a conductive circuit 36, the conductive circuit 36 is connected between the controller 34 and the switch 32, and a signal outputted by the first sensor 33 and indicating a detection result controls the conductive circuit 36 to be open or closed.

For example, when the first sensor 33 detects that the first region 3101 is obstructed, the signal outputted by the first sensor 33 and indicating the detection result causes the conductive circuit 36 to be closed, and in this case: when the switch 32 is in a triggered state, the controller 34 outputs the control signal for allowing movement; and when the switch 32 is in an untriggered state, the controller 34 does not output the control signal for allowing movement.

For another example, when the first sensor 33 does not detect that the first region 3101 is obstructed, the signal outputted by the first sensor 33 and indicating the detection result causes the conductive circuit 36 to be open, and in this case, the signal outputted by the switch 32 and indicating the state of the switch cannot be sent to the controller 34, so that the controller 34 does not output the control signal for allowing movement regardless of whether the switch 32 is in the triggered state.

In the embodiment shown by (B) of FIG. 4, the signal outputted by the first sensor 33 and indicating the detection result is introduced via the conductive circuit 36, so that the impact on wiring of an input pin of the controller 34 is small, modification is facilitated, and the impact on control logic of the controller 34 is small.

It should be noted that the connecting circuit shown in FIG. 4 is merely an example, and the present application is not limited thereto. The connecting circuit of the first sensor 33, the switch 32, and the controller 34 may alternatively have another form.

As shown in FIG. 3, in the present application, the pedal assembly 300 may further have the protective cover 35. The protective cover 35 can protect one side of the pedal 31 facing the pedaling surface 310A. An accommodating portion 350 is formed between the protective cover 35 and the pedaling surface 310A of the pedal 31, and a part of the body of the operator (for example, a foot) stretches into the accommodating portion 350 and then steps on or presses the pedaling surface 310A of the pedal 31. The protective cover 35 can reduce the possibility that a part of the body of the operator (for example, a foot) unintentionally steps on or presses the pedal 31, for example, in some cases, a part of the body of the operator (for example, a foot) steps on or presses the protective cover 35, thereby preventing the pedal 31 from being unintentionally stepped on or pressed. In the present application, the protective cover 35 is an optional component, i.e., the pedal assembly 300 may or may not have the protective cover 35.

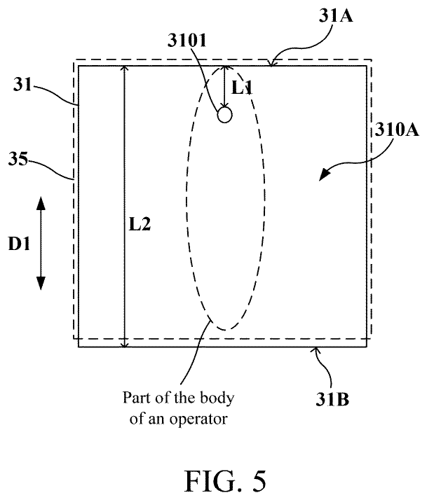

FIG. 5 is a schematic diagram of the pedal. As shown in FIG. 5, the first region 3101 may be a position where a part of the body of the operator (for example, a foot) intentionally steps on or presses the pedal 31. When a part of the body of the operator (for example, a foot) intentionally steps on or presses the pedal 31, the part of the body may step on or press the inner side of the pedal assembly as much as possible, to obstruct a position of the pedaling surface 310A of the pedal 31 that is close to the first end portion 31A. Therefore, in some examples, the first region 3101 may be a position close to the first end portion 31A. For example, in the first direction D1, the distance L1 between the first region 3101 of the pedaling surface 310A of the pedal 31 and the first end portion 31A is smaller than ⅕ of a dimension L2 of the pedal 31 in the first direction D1. In addition, ⅕ herein is merely an example, and another value may be used.

In the description of the embodiments described above, only a case in which one pedal 31 is provided with one first sensor 33 is shown. The present application is not limited thereto. For example, two or more first regions 3101 at different positions are disposed on a surface of one pedal 31, and each first region 3101 has a corresponding first sensor 33. Therefore, the one pedal 31 is provided with two or more first sensors 33, and the two or more first sensors 33 may be connected in parallel, that is, as long as one of the two or more first sensors 33 outputs a signal indicating that a first position is obstructed, it may be considered that a part of the body of the operator (for example, a foot) is intentionally stepping on or pressing the pedal 31.

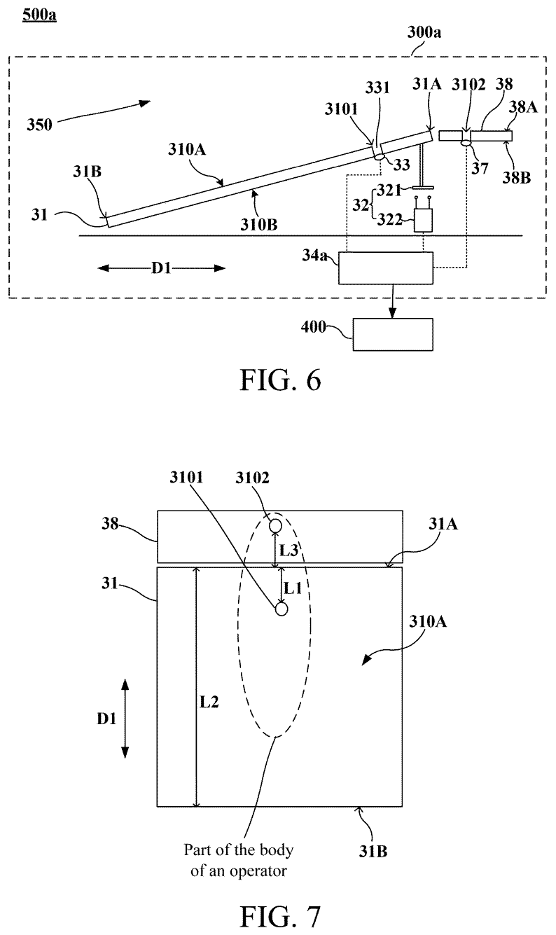

FIG. 6 is another schematic diagram of a pedal assembly according to an embodiment of the present application. As shown in FIG. 6, the pedal assembly 300a includes: a pedal 31, a switch 32, a first sensor 33, a second sensor 37, and a controller 34a.

For descriptions of the pedal 31, the switch 32, and the first sensor 33 in FIG. 6, reference may be made to corresponding descriptions of the pedal 31, the switch 32, and the first sensor 33 in FIG. 3. The following describes the second sensor 37 and the controller 34a in FIG. 6. As shown in FIG. 6, the second sensor 37 detects whether a second region 3102 outside the pedal 31 is obstructed.

FIG. 7 is a schematic diagram of the pedal in FIG. 6. As shown in FIG. 6 and FIG. 7, there is a frame 38 outside the pedal 31. The second region 3102 may be located on the frame 38. In the first direction D1, the second region 3102 is closer to the inner side of the pedal assembly 300a than the first region 3101. For example, in the first direction D1, the distance between the second region 3102 and the first end portion 31A of the pedal 31 may be L3, and L3 may be less than or equal to 1/10 of the dimension L2 of the pedal 31 in the first direction D1.

In the present application, the second region 3102 is located outside the pedal 31. Therefore, when the pedal assembly 300a does not have the protective cover 35 shown in FIG. 3, the second region 3102 is obstructed, which may correspond to a scenario in which a part of the body of the operator (for example, a foot) unintentionally steps on or presses the pedal 31. Therefore, by detecting, by the second sensor 37, whether the second region 3102 outside the pedal 31 is obstructed, it is possible to more reliably determine that a part of the body of the operator (for example, a foot) is unintentionally stepping on or pressing the pedal 31, thereby preventing unexpected movement of the external mechanism 400.

In the present application, the second sensor 37 may be disposed on a side of a back surface 38B of the frame 38 to detect, via a hole 381 in the frame 38, whether the second region 3102 is obstructed. The back surface 38B faces one side of the switch 32, and may serve as a non-pedaling surface, an inner surface, or a lower surface of the frame 38. In addition, the second sensor 37 may alternatively be disposed on a side of a pedaling surface 38A of the frame 38. For example, the second sensor 37 is disposed on the pedaling surface 38A of the frame 38. The pedaling surface 38A is an upper surface or an outer surface of the frame 38, to be stepped on or pressed by a part of the body of the operator (for example, a foot).

The second sensor 37 may be a photoelectric sensor, a capacitive sensor, an image recognition sensor, a pressure sensor, a magnetic sensor, or the like. For the description of the operating principle of the second sensor 37, reference may be made to the description of the operating principle of the first sensor 33.

In the pedal assembly 300a shown in FIG. 6, the controller 34a outputs a control signal based on a detection result of the first sensor 33, a detection result of the second sensor 37, and a state of the switch 32, wherein the control signal may be used to control the external mechanism 400 to move or not to move. Therefore, the controller 34a outputs the control signal not only based on the state of the switch 32 and the detection result of the first sensor 33, but also taking into consideration the detection result of the second sensor 37 (i.e., whether the second region 3102 is obstructed), so that it is possible to avoid unexpected movement of the external mechanism 400 occurring when a part of the body of the operator (for example, a foot) unintentionally touches the pedal 31 and the outside of the pedal 31, thereby improving the stability and safety of the movement of the external mechanism 400.

For example, in some scenarios, when the switch 32 is in the triggered state, the first sensor 33 detects that the first region 3101 is obstructed, and the second sensor 37 detects that the second region 3102 is not obstructed, the controller 34a outputs a control signal for allowing movement, wherein the control signal for allowing movement may control the external mechanism 400 to move. These scenarios correspond to a case in which a part of the body of the operator (for example, a foot) intentionally steps on or presses the pedal 31 and the pedal 31 leaves the predetermined position. In this case, the part of the body of the operator (for example, a foot) obstructs the first region 3101 but does not obstruct the second region 3102.

For another example, in some scenarios, when the switch 32 is in the triggered state, the first sensor 33 detects that the first region 3101 is obstructed, and the second sensor 37 detects that the second region 3102 is also obstructed, the controller 34a does not output a control signal for allowing movement, wherein not outputting a control signal for allowing movement may be not outputting any signal, thereby causing the external mechanism 400 to remain in a stationary state, or outputting a control signal to cause the external mechanism to remain in the stationary state. These scenarios correspond to a case in which a part of the body of the operator (for example, a foot) unintentionally steps on or presses the pedal 31 and the second region 3102 and the pedal 31 leaves the predetermined position. In this case, the part of the body of the operator (for example, a foot) obstructs the first region 3101 and the second region 3102.

For still another example, in some other scenarios, when the switch 32 is in the triggered state and the first sensor 33 detects that the first region 3101 is not obstructed, the controller 34a does not output a control signal for allowing movement. These scenarios correspond to a case in which a part of the body of the operator (for example, a foot) unintentionally steps on or presses the pedal 31 and the pedal 31 leaves the predetermined position. In this case, the part of the body of the operator (for example, a foot) does not obstruct the first region 3101 or the second region 3102.

For yet another example, in some scenarios, when the switch 32 is in an untriggered state, the controller 34a does not output a control signal for allowing movement. These scenarios correspond to a case in which a part of the body of the operator (for example, a foot) does not step on or press the pedal 31 or steps on or presses the pedal 31 but the pedal 31 does not leave the predetermined position.

In the present application, the external mechanism 400 controlled by the controller 34a may include a scanning bed that, for example, is applied to a medical imaging system, wherein the medical imaging system has, for example, a CT (computed tomography) imaging device, or a PET (positron emission tomography)-CT, a magnetic resonance imaging (MRI), or any other suitable medical imaging device. Therefore, the scanning bed may also be referred to as a medical imaging scanning bed. Therefore, the present application may provide a medical imaging scanning bed assembly 500a including the external mechanism 400 and the pedal assembly 300a.

For example, in the medical imaging scanning bed assembly 500a, portions other than the controller 34a in the pedal assembly 300a may correspond to the pedal assembly 2072 of FIG. 2. The controller 34a of the pedal assembly 300a corresponds to the computer 205 of FIG. 2, and the external mechanism 400 may correspond to the patient table 102 of FIG. 2, i.e., a medical imaging scanning bed. For example, the controller 34a (corresponding to the computer 205 of FIG. 2) of the pedal assembly 300a outputs a control signal based on the detection result of the first sensor 33, the detection result of the second sensor 37, and the state of the switch 32, the control signal is inputted to the patient table motor controller 209 of FIG. 2, and the patient table motor controller 209 outputs a signal to the patient table 102 (corresponding to the external mechanism 400 of FIG. 3), so that the patient table 102 moves (e.g., lifts or lowers) or does not move.

In addition, in another example, the controller 34a of the pedal assembly 300a may alternatively correspond to both the computer 205 and the patient table motor controller 209 of FIG. 2, that is, the controller 34a (corresponding to both the computer 205 and the patient table motor controller 209 of FIG. 2) of the pedal assembly 300a outputs a control signal based on the detection result of the first sensor 33, the detection result of the second sensor 37, and the state of the switch 32, and the control signal is inputted to the patient table 102 (corresponding to the external mechanism 400 of FIG. 3) of FIG. 2, so that the patient table 102 moves (e.g., lifts or lowers) or does not move.

In the present application, the pedal assembly 300a being applied to the medical imaging scanning bed assembly 500a is used as an example for description. However, the description is also applicable to a case in which the pedal assembly 300a is applied to another system other than the medical imaging scanning bed assembly 500a.

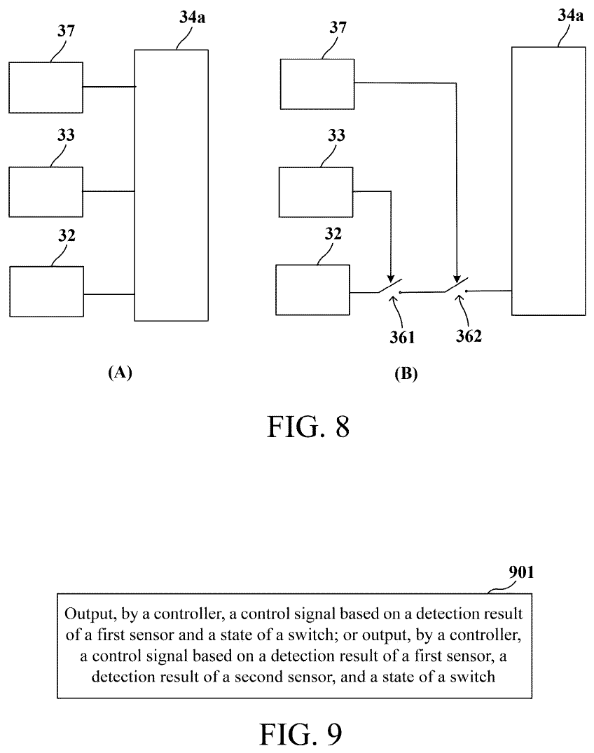

FIG. 8 is a schematic diagram of a connecting circuit of the first sensor 33, the second sensor 37, the switch 32, and the controller 34a according to the present application. (A) and (B) of FIG. 8 may be different embodiments.

In the embodiment shown by (A) of FIG. 8: the first sensor 33 and the second sensor 37 are connected to the controller 34a, the first sensor 33 outputs to the controller 34a a signal indicating a detection result of whether a first position is obstructed, and the second sensor 37 outputs to the controller 34a a signal indicating a detection result of whether a second position is obstructed. The switch 32 is connected to the controller 34a, and the switch 32 outputs to the controller 34a a signal indicating a state of the switch. The controller 34a outputs a control signal based on the signal outputted by the first sensor 33 and indicating the detection result, the signal outputted by the second sensor 37 and indicating the detection result, and the signal outputted by the switch 32 and indicating the state of the switch. For logic for the controller 34a to output the control signal, reference may be made to the description described above with respect to FIG. 6. The connecting circuit in the embodiment shown by (A) of FIG. 8 is simple.

In the embodiment shown by (B) of FIG. 8: the first sensor 33 is connected to a first conductive circuit 361, the second sensor 37 is connected to a second conductive circuit 362, the first conductive circuit 361 and the second conductive circuit 362 are connected in series between the controller 34a and the switch 32, a signal outputted by the first sensor 33 and indicating a detection result controls the first conductive circuit 361 to be open or closed, and a signal outputted by the second sensor 37 and indicating a detection result controls the second conductive circuit 362 to be open or closed.

For example, when the first sensor 33 detects that the first region 3101 is obstructed, the signal outputted by the first sensor 33 and indicating the detection result causes the first conductive circuit 361 to be closed, when the second sensor 37 does not detect that the second region 3102 is obstructed, the signal outputted by the second sensor 37 and indicating the detection result causes the second conductive circuit 362 to be closed, and in this case: when the switch 32 is in the triggered state, the controller 34a outputs the control signal for allowing movement, and when the switch 32 is in the untriggered state, the controller 34a does not output the control signal for allowing movement.

For another example, when the first sensor 33 detects that the first region 3101 is obstructed, the signal outputted by the first sensor 33 and indicating the detection result causes the first conductive circuit 361 to be closed, when the second sensor 37 detects that the second region 3102 is obstructed, the signal outputted by the second sensor 37 and indicating the detection result causes the second conductive circuit 362 to be open, and in this case, the signal outputted by the switch 32 and indicating the state of the switch cannot be sent to the controller 34a, so that the controller 34a does not output the control signal for allowing movement regardless of whether the switch 32 is in the triggered state.

For still another example, when the first sensor 33 does not detect that the first region 3101 is obstructed, the signal outputted by the first sensor 33 and indicating the detection result causes the first conductive circuit 361 to be open, when the second sensor 37 does not detect that the second region 3102 is obstructed, the signal outputted by the second sensor 37 and indicating the detection result causes the second conductive circuit 362 to be closed, and in this case, the signal outputted by the switch 32 and indicating the state of the switch cannot be sent to the controller 34a, so that the controller 34a does not output the control signal for allowing movement regardless of whether the switch 32 is in the triggered state.

In the embodiment shown by (B) of FIG. 8, the signal outputted by the first sensor 33 and indicating the detection result and the signal outputted by the second sensor 37 and indicating the detection result are introduced via the first conductive circuit 361 and the second conductive circuit 362, respectively, so that the impact on wiring of an input pin of the controller 34a is small, modification is facilitated, and the impact on control logic of the controller 34a is small.

It should be noted that the connecting circuit shown in FIG. 8 is merely an example, and the present application is not limited thereto. The connecting circuit of the first sensor 33, the second sensor 37, the switch 32, and the controller 34a may alternatively have another form.

For simplicity, the figures only exemplarily illustrate the connection relationship or signal direction between various components or modules, but it should be clear to those skilled in the art that various related technologies such as bus connection can be used. The various components or modules described above can be implemented by means of hardware such as a processor or a memory, etc. The embodiments of the present application are not limited thereto.

The foregoing embodiments merely provide illustrative descriptions of the embodiments of the present application. However, the present application is not limited thereto, and appropriate variations may be made on the basis of the foregoing embodiments. For example, each of the embodiments described above may be used independently, or one or more among the foregoing embodiments may be combined.

According to the embodiments of the first aspect of the present application, whether a predetermined position on a surface of a pedal is obstructed is detected by means of a first sensor, and a control signal is outputted based on a detection result of the first sensor and a state of a switch, wherein the control signal may be used to control the operation of a mechanism such as a scanning bed. For example, when the operator unintentionally steps on the pedal to cause the switch to be in a triggered state, the predetermined position on the surface of the pedal is not obstructed by a foot or shoe of the operator, and the controller does not output a control signal to cause the mechanism such as the scanning bed to operate. In this way, unexpected movement of the mechanism such as the scanning bed is avoided.

In addition, the first sensor is disposed in a space on a side of a back surface of the pedal, to avoid enlargement and complexity of the pedal assembly, so that there is little impact on the use of the pedal, and the scope of application of the pedal assembly can be expanded.

In addition, in the present application, a second sensor is further utilized to detect whether a position outside the pedal is obstructed, and the control signal is outputted based on the detection result. Therefore, in a scenario in which the pedal does not have a protective cover, unexpected movement of a mechanism such as a scanning bed due to mistaken stepping or touching by the operator can be more reliably avoided.

Provided in the embodiments of the second aspect of the present application is a control method based on a pedal assembly, to control the pedal assembly according to the embodiments of the first aspect. The content of the embodiments of the second aspect the same as that of the embodiments of the first aspect is not repeated herein.

FIG. 9 is a schematic diagram of a control method based on a pedal assembly according to an embodiment of the present application. As shown in FIG. 9, the control method based on a pedal assembly according to this embodiment of the present disclosure includes outputting, by a controller, a control signal based on a detection result of a first sensor and a state of a switch; or outputting, by a controller, a control signal based on a detection result of a first sensor, a detection result of a second sensor, and a state of a switch in step 901. For a detailed description of operation 901, reference may be made to relevant descriptions of the controller 34 or 34a in the embodiments of the first aspect.

The foregoing embodiments merely provide illustrative descriptions of the embodiments of the present application. However, the present application is not limited thereto, and appropriate variations may be made on the basis of the foregoing embodiments. For example, each of the embodiments described above may be used independently, or one or more among the foregoing embodiments may be combined.

According to the embodiments of the second aspect of the present application, unexpected movement of a mechanism such as a scanning bed due to mistaken stepping or touching by an operator can be avoided.

An embodiment of the present application further provides a computer-readable program or program product, wherein when the program is executed in an electronic device, the program causes a computer to execute, in the electronic device, the control method based on a pedal assembly as described in the embodiments of the second aspect.

An embodiment of the present application further provides a storage medium storing a computer-readable program, wherein the computer-readable program causes a computer to execute, in an electronic device, the control method based on a pedal assembly as described in the embodiments of the second aspect.

The above apparatus and method of the present application can be implemented by hardware, or can be implemented by hardware in combination with software. The present application relates to such a computer-readable program that when executed by a logic component, the program causes the logic component to implement the foregoing apparatus or a constituent component, or causes the logic component to implement various methods or steps as described above. The present application further relates to a storage medium for storing the above program, such as a hard disk, a disk, an optical disk, a DVD, a flash memory, etc.

The method/apparatus described in view of the embodiments of the present application may be directly embodied as hardware, a software module executed by a processor, or a combination of the two. For example, one or more of the functional block diagrams and/or one or more combinations of the functional block diagrams shown in the drawings may correspond to either respective software modules or respective hardware modules of a computer program flow. The foregoing software modules may respectively correspond to the steps shown in the figures. The foregoing hardware modules can be implemented, for example, by firming the software modules using a field-programmable gate array (FPGA).

The software modules may be located in a RAM, a flash memory, a ROM, an EPROM, an EEPROM, a register, a hard disk, a portable storage disk, a CD-ROM, or any other form of storage medium known in the art. The storage medium may be coupled to a processor, so that the processor can read information from the storage medium and can write information into the storage medium. Alternatively, the storage medium may be a constituent component of the processor. The processor and the storage medium may be located in an ASIC. The software module may be stored in a memory of a mobile terminal, and may also be stored in a memory card that can be inserted into a mobile terminal. For example, if a device (such as a mobile terminal) uses a large-capacity MEGA-SIM card or a large-capacity flash memory apparatus, the software modules can be stored in the MEGA-SIM card or the large-capacity flash memory apparatus.

One or more of the functional blocks and/or one or more combinations of the functional blocks shown in the accompanying drawings may be implemented as a general-purpose processor, a digital signal processor (DSP), an application-specific integrated circuit (ASIC), a field-programmable gate array (FPGA) or other programmable logic devices, discrete gate or transistor logic devices, a discrete hardware assembly, or any appropriate combination thereof for implementing the functions described in the present application. The one or more functional blocks and/or the one or more combinations of the functional blocks shown in the accompanying drawings may also be implemented as a combination of computing devices, such as a combination of a DSP and a microprocessor, multiple microprocessors, one or more microprocessors in communication combination with a DSP, or any other such configuration.

The present application is described above with reference to specific embodiments. However, it should be clear to those skilled in the art that the foregoing description is merely illustrative and is not intended to limit the scope of protection of the present application. Various variations and modifications may be made by those skilled in the art according to the principle of the present application, and said variations and modifications also fall within the scope of the present application.

1. A pedal assembly, characterized by comprising:
a pedal, having a first end portion close to an inner side of the pedal assembly in a first direction and a second end portion away from the inner side of the pedal assembly;
a switch, connected to the pedal, the switch being in a triggered state when the pedal leaves a predetermined position in a second direction;
a first sensor, detecting, via a through hole on a pedaling surface of the pedal, whether a first region of the pedaling surface of the pedal is obstructed; and
a controller, outputting a control signal based on a detection result of the first sensor and a state of the switch.

2. The pedal assembly according to claim 1, wherein
the distance between the first region and the first end portion in the first direction is less than ⅕ of a dimension of the pedal in the first direction.

3. The pedal assembly according to claim 1, wherein
when the switch is in the triggered state and the first sensor detects that the first region is obstructed, the controller outputs a control signal for allowing movement; or
when the switch is in the triggered state and the first sensor detects that the first region is not obstructed, the controller does not output a control signal for allowing movement.

4. The pedal assembly according to claim 1, wherein
the first sensor is connected to the controller, and the first sensor outputs a signal indicating a detection result to the controller; or
the first sensor is connected to a conductive circuit connected between the controller and the switch, and a signal outputted by the first sensor and indicating a detection result controls the conductive circuit to be open or closed.

5. The pedal assembly according to claim 1, wherein
the pedal assembly further comprises:
a second sensor, detecting whether a second region outside the pedal is obstructed,
wherein the controller outputs the control signal based on the detection result of the first sensor, a detection result of the second sensor, and the state of the switch.

6. The pedal assembly according to claim 5, wherein
in the first direction, the second region is closer to the inner side of the pedal assembly than the first region.

7. The pedal assembly according to claim 5, wherein
when the switch is in the triggered state, the first sensor detects that the first region is obstructed, and the second sensor detects that the second region is not obstructed, the controller outputs a control signal for allowing movement; or
when the switch is in the triggered state, the first sensor detects that the first region is obstructed, and the second sensor detects that the second region is obstructed, the controller does not output a control signal for allowing movement; or
when the switch is in the triggered state and the first sensor detects that the first region is not obstructed, the controller does not output a control signal for allowing movement.

8. The pedal assembly according to claim 5, wherein
the first sensor and the second sensor are connected to the controller, the first sensor outputs a signal indicating a detection result to the controller, and the second sensor outputs a signal indicating a detection result to the controller; or
the first sensor is connected to a first conductive circuit, the second sensor is connected to a second conductive circuit, the first conductive circuit and the second conductive circuit are connected in series between the controller and the switch, a signal outputted by the first sensor and indicating a detection result controls the first conductive circuit to be open or closed, and a signal outputted by the second sensor and indicating a detection result controls the second conductive circuit to be open or closed.

9. The pedal assembly according to claim 1, wherein
the first sensor is mounted on a back surface of the pedal, the back surface being a surface opposite to the pedaling surface of the pedal; or
the first sensor is mounted outside the pedal, and the distance between the first sensor and a back surface of the pedal is smaller than the distance between the first sensor and the pedaling surface of the pedal.

10. A control method based on a pedal assembly, characterized in that the pedal assembly comprises:
a pedal, having a first end portion close to an inner side of the pedal assembly in a first direction and a second end portion away from the inner side of the pedal assembly;
a switch, connected to the pedal, the switch being in a triggered state when the pedal leaves a predetermined position in a second direction;
a first sensor, detecting, via a through hole on a pedaling surface of the pedal, whether a first region of the pedaling surface of the pedal is obstructed; and
a controller; and
the control method comprises:
outputting, by the controller, a control signal based on a detection result of the first sensor and a state of the switch.

11. The control method according to claim 10, wherein
when the switch is in the triggered state and the first sensor detects that the first region is obstructed, the controller outputs a control signal for allowing movement; or
when the switch is in the triggered state and the first sensor detects that the first region is not obstructed, the controller does not output a control signal for allowing movement.

12. The control method according to claim 10, wherein
the pedal assembly further comprises:
a second sensor, detecting whether a second region outside the pedal is obstructed,
wherein the control method comprises:
outputting, by the controller, the control signal based on the detection result of the first sensor, a detection result of the second sensor, and the state of the switch.

13. The control method according to claim 10, wherein
when the switch is in the triggered state, the first sensor detects that the first region is obstructed, and the second sensor detects that the second region is not obstructed, the controller outputs a control signal for allowing movement; or
when the switch is in the triggered state, the first sensor detects that the first region is obstructed, and the second sensor detects that the second region is obstructed, the controller does not output a control signal for allowing movement; or
when the switch is in the triggered state and the first sensor detects that the first region is not obstructed, the controller does not output a control signal for allowing movement.
